(19) United States
(12) Patent Application Publication
(10) Pub. No.: US 2004/0171118 A1
(43) Pub. Date: Sep. 2, 2004

(54) Methods for directing DNA methylation in mammalian cells using homologous short double stranded RNAs

(75) Inventors: John J. Rossi, Altma Loma, CA (US); Daniela Castanotto, Altadena, CA (US); Gerd Pfeiffer, Bradbury, CA (US); Stella Tommassi, South Pasadena, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 10/776,635

(22) Filed: Feb. 12, 2004

Related U.S. Application Data
(60) Provisional application No. 60/447,013, filed on Feb. 13, 2003.

(51) Int. Cl. C12P 21/06

(52) U.S. Cl. 435/69.1

(57) ABSTRACT

The invention provides methods for methylating a gene of interest in a cell. The methods include exposing a mammalian cell to an siRNA molecule which is specific for a gene of interest in the cell. The methods also include introducing into the cell DNA sequences encoding a sense strand and an antisense strand of an siRNA which is specific for the gene of interest. The siRNA directs methylation of the gene of interest.

This application claims priority to co-pending U.S. Provisional Application No. 60/447,013, filed Feb. 13, 2003, which is incorporated herein by reference.

The invention described herein was made with Government support under grant numbers AI29329 and AI42552 from the National Institutes of Health. Accordingly, the United States Government may have certain rights in this invention.

### FIELD OF THE INVENTION

The present invention relates to DNA methylation and interfering RNA.

### BACKGROUND OF THE INVENTION

Throughout this application, various publications are referenced within parentheses. Disclosures of these publications in their entireties are hereby incorporated into this application to more fully describe the state of the art to which this invention pertains. Full bibliographic citations for the references may be found listed immediately preceding the claims.

RNA silencing is a eukaryotic genome defense mechanism that involves processing of longer duplex RNAs into short double stranded RNAs (21 to 26 nucleotides in length). These short RNAs are called short interfering RNAs (siRNAs). It has been recently described for plants that two separate classes of siRNAs exist: short (21 base pairs) and long (26 base pairs). In plants, the short siRNAs direct target specific degradation of mRNAs whereas the longer RNAs direct genome and target specific methylation of DNA, resulting in genetic silencing (Hamilton, et al.). Hamilton, et al. indicates that the short siRNAs do not correlate with systemic signaling or methylation.

RNA interference (RNAi), which includes the process in which double stranded RNA (dsRNA) induces the post-transcriptional degradation of homologous transcripts (via short siRNAs, as described above), is typically initiated by exposing cells to dsRNA either via transfection or endogenous expression. As indicated, double-stranded RNAs are processed into the siRNAs. (Elbashir, S. M., et al., 2001a; Elbashir, S. M., et al., 2001b). These siRNAs form a complex known as the RNA Induced Silencing Complex or RISC (Bernstein, E., et al.; Hammond, S. M., et al.), which functions in homologous target RNA destruction. In mammalian systems, the sequence specific RNAi effect can be observed by introduction of siRNAs either via transfection or endogenous expression of 21-23 base transcripts or longer hairpin RNAs. Use of siRNAs evades the dsRNA induced interferon and PKR pathways that lead to non-specific inhibition of gene expression. (Elbashir, et al., 2001).

Recently, several groups have demonstrated that siRNAs can be effectively transcribed by Pol III promoters in human cells, and elicit target specific mRNA degradation. (Lee, N. S., et al., 2002; Miyagishi, M., et al., 2002; Paul, C. P., et al., 2002; Brummelkamp, T. R., et al., 2002; Ketting, R. F., et al., 2001). These siRNA encoded genes have been transiently transfected into human cells using plasmid or episomal viral backbones for delivery. Transient siRNA expression can be useful for rapid phenotypic determinations preliminary to making constructs designed to obtain long term siRNA expression. Of particular interest is the fact that not all sites along a given mRNA are equally sensitive to siRNA-mediated downregulation. (Elbashir, S. M., et al., 2001; Lee, N. S., et al., 2001; Yu, J. Y., et al., 2002; Holen, T, et al., 2002). There are at this time very few rules governing siRNA target site selection for a given mRNA target. It is therefore important to be able to rapidly screen potential target sequences to identify a sequence or sequences susceptible to siRNA mediated degradation. Initial attempts at addressing this problem have taken advantage of an oligonucleotide/RNAseH procedure in cell extracts on native mRNA transcripts designed to identify ribozyme accessible sites. This approach has been applied to siRNA site accessibility as well. (Lee, N. S., et al., 2001). However, this process can be time consuming, and in the end it is still necessary to synthesize the siRNA genes for final testing.

Co-pending U.S. Provisional Application No. 60/408,298, filed on Sep. 6, 2002, incorporated herein by reference, is directed to an amplification-based approach (e.g., PCR) for rapidly synthesizing promoter-containing siRNA gene constructs and subsequently transfecting them into cells, permitting rapid screening of potential target sequences susceptible to siRNA mediated degradation.

Co-pending U.S. Provisional Application No. 60/356,127, filed on Feb. 14, 2002, incorporated herein by reference, is directed to methods for producing double-stranded, interfering RNA molecules in mammalian cells. These methods overcome prior limitations to the use of siRNA as a therapeutic agent in vertebrate cells, including the need for short, highly defined RNAs to be delivered to target cells other than through the use of synthetic, duplexed RNAs delivered exogenously to cells.

In contrast to post-transcriptional silencing involving degradation of mRNA by short siRNAs, the use of long siRNAs to methylate DNA has been shown to provide an alternate means of gene silencing in plants. (Hamilton, et al.). In higher order eukaryotes, DNA is methylated at cytosines located 5′ to guanosine in the CpG dinucleotide. This modification has important regulatory effects on gene expression, especially when involving CpG-rich areas known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosomes of females. Aberrant methylation of normally unmethylated CpG islands has been documented as a relatively frequent event in immortalized and transformed cells and has been associated with transcriptional inactivation of defined tumor suppressor genes in human cancers. In this last situation, promoter region hypermethylation stands as an alternative to coding region mutations in eliminating tumor suppression gene function. (Herman, et al.).

There exists a need in the art, however, for methods to modify gene function in mammals by directing methylation of a target gene, including the promoter region of the target gene, in mammalian and preferably human cells.

### SUMMARY OF THE INVENTION

The present invention provides a method for directing DNA methylation in mammalian cells. It has been found that siRNAs (from about 21-28 nucleotides) can be used to direct methylation of DNA in mammalian cells.

In one aspect, the invention provides a method for methylating a gene of interest in a mammalian cell comprising exposing the cell to a siRNA, which is specific for a target sequence on the gene.

In another aspect, the invention provides a method for methylating a gene of interest in a mammalian cell comprising introducing into the cell DNA sequences encoding a sense strand and an antisense strand of an siRNA which is specific for a target sequence on the gene, preferably under conditions permitting expression of the siRNA in the cell, and wherein the siRNA directs methylation of the gene of interest.

In accordance with the present invention, DNA methylation can be directed to any region that can be methylated. In a preferred embodiment, the region includes a CpG island. In another embodiment, the region is a promoter region.

### BRIEF DESCRIPTION OF THE FIGURES

FIG. 1 is a schematic representation of a PCR strategy used to yield U6 transcription cassettes expressing siRNAs. The 5′ PCR primer is complementary to the 5′ end of the U6 promoter and is standard for all PCR reactions. A) The 3′ PCR primer is complementary to sequences at the 3′ end of the U6 promoter and is followed by the sense or antisense sequences, a stretch of four to six deoxyadenosines (Ter) and an additional stuffer-Tag sequence. The adenosines are the termination signal for the U6 Pol III promoter; therefore, any sequence added after this signal will not be transcribed by the Pol III polymerase and will not be part of the siRNA. B) The sense and antisense sequences are linked by a 9 nt loop and are inserted in the cassette by a two-step PCR reaction. C) The sense and antisense sequences linked by a 9-nucleotide loop and followed by the stretch of adenosines and by the Tag sequences are included in a single 3′ primer. D) Complete PCR expression cassette obtained by the PCR reaction. To amplify and identify functional siRNAs from the transfected cells, or to increase the yield of the PCR product shown in D, a nested PCR can be performed using the universal 5′ U6 primer and a 3′ primer complementary to the Tag sequence, as indicated in the figure.

FIG. 2 shows the results of a methylation specific PCR (MSP) analysis of the RASSF1A promoter in siRNA transfected cells.

FIG. 3 illustrates DNA sequences of the RASSF1A promoter that became methylated in siRNA transfected cells.

FIG. 4 shows the results of RASSF1A intracellular expression in stable clones and cell populations (siPR28) transfected with specific shRNAs.

FIG. 5 shows the results of RASSF1A intracellular expression in stable clones transfected with 28 nucleotides shRNAs.

FIG. 6 shows the results of RNA down-regulation by shRNA directed against the RASSF1A promoter, as detected by transient transfections and quantitative PCR.

### DETAILED DESCRIPTION OF THE INVENTION

The present invention provides methods for directing DNA methylation of a target sequence in a gene using siRNAs.

The siRNAs may be produced in any number of ways. The siRNAs may be chemically produced, or expressed in cells, and subsequently purified. The siRNAs also may be expressed directly in mammalian, preferably human, cells containing the gene of interest to be methylated.

The siRNA molecule may have different forms, including a single strand, a paired double strand or a hairpin (shRNA).

In a preferred embodiment, the invention provides a method for methylating a gene of interest in a mammalian cell comprising introducing into the cell DNA sequences encoding a sense strand and an antisense strand of an siRNA, which is specific for a target sequence in the gene of interest, preferably under conditions permitting expression of the siRNA in the cell, and wherein the siRNA directs methylation of said gene of interest. In an embodiment, methylation is directed to a sequence in the promoter region of the gene. Alternately, methylation is directed to a sequence in the coding region. Target sequences can be any sequence in a gene that has the potential for methylation. In a preferred embodiment, the target sequences contain CpG islands. The directed methylation can lead to activation or inactivation of the gene.

Once a target sequence or sequences have been identified for methylation in accordance with the invention, the appropriate siRNA can be produced, for example, either sythetically or by expression in cells.

In a preferred embodiment, DNA sequences for encoding the sense and antisense strands of the siRNA molecule to be expressed directly in mammalian cells can be produced by methods known in the art and described herein.

Possible target sequences include those found on cellular or infectious agent genes (viral, etc.) or any gene whose activation or inactivation is desired. The gene can be a RASSF1A gene.

In accordance with a preferred embodiment of the present invention, DNA sequences encoding a sense strand and an antisense strand of a siRNA specific for a target sequence of a gene are introduced into mammalian cells for expression. To target more than one sequence in the gene (such as different promoter region sequences and/or coding region sequences), separate siRNA-encoding DNA sequences specific to each targeted gene sequence can be introduced simultaneously into the cell.

In accordance with another embodiment, mammalian cells may be exposed to multiple siRNAs that target multiple sequences in the gene.

The siRNA molecules generally contain about 19 to about 28 base pairs, and preferably are designed to cause methylation of the targeted gene sequence. In one embodiment, the siRNA molecules contain about 19-23 base pairs, and preferably about 21 base pairs. In another embodiment, the siRNA molecules contain about 24-28 base pairs, and preferably about 26 base pairs. Individual siRNA molecules also may be in the form of single strands, as well as paired double strands (“sense” and “antisense”) and may include secondary structure such as a hairpin loop. Individual siRNA molecules could also be delivered as precursor molecules, which are subsequently altered to give rise to active molecules. Examples of siRNA molecules in the form of single strands include a single stranded anti-sense siRNA against a non-transcribed region of a DNA sequence (e.g. a promoter region).

In a preferred embodiment, the DNA sequences encoding the sense and antisense strands of the siRNA molecule can be generated by PCR. In another preferred embodiment, the siRNA encoding DNA is cloned into a vector, such as a plasmid or viral vector, to facilitate transfer into mammals. In another embodiment, siRNA molecules may be synthesized using chemical or enzymatic means.

The sense and antisense strands of the siRNA can be expressed independently or linked preferably by a 9 nucleotides loop (different size loops could also be used). The Pol III cassettes expressing the single stranded form of the siRNA can be transfected as PCR products, or cloned into separate or a single expression vector. Similarly, the siRNAs constructed as a stem loop can be transfected in the form of a PCR product or cloned into an expression vector.

To facilitate nuclear retention and increase the level of methylation, the sense and antisense strands of the siRNA molecule may be expressed in a single stranded form, for example as a stem loop structure, as described above. Alternatively, or in concomitance, the factor(s) involved in the active cellular transport of siRNA's, such as Exportin 5, may be downregulated employing synthetic siRNA, antisense, ribozymes, or any other nucleic acid, antibody or drug, proven to be effective in downregulating the gene(s) of interest.

The term “introducing” encompasses a variety of methods of introducing DNA into a cell, either in vitro or in vivo. Such methods include transformation, transduction, transfection, and infection. Vectors are useful and preferred agents for introducing DNA encoding the siRNA molecules into cells. The introducing may be accomplished using at least one vector. Possible vectors include plasmid vectors and viral vectors. Viral vectors include retroviral vectors, lentiviral vectors, or other vectors such as adenoviral vectors or adeno-associated vectors.

In one embodiment, the DNA sequences are included in separate vectors, while in another embodiment, the DNA sequences are included in the same vector. The DNA sequences may be inserted into the same vector as a multiple cassettes unit.

Alternate delivery of siRNA molecules or DNA encoding siRNA molecules into cells or tissues may also be used in the present invention, including liposomes, chemical solvents, electroporation, viral vectors, as well as other delivery systems known in the art.

Suitable promoters include those promoters that promote expression of the interfering RNA molecules once operatively associated or linked with sequences encoding the RNA molecules. Such promoters include cellular promoters and viral promoters, as known in the art. In one embodiment, the promoter is an RNA Pol III promoter, which preferably is located immediately upstream of the DNA sequences encoding the interfering RNA molecule. Various viral promoters may be used, including, but not limited to, the viral LTR, as well as adenovirus, SV40, and CMV promoters, as known in the art.

In a preferred embodiment, the invention uses a mammalian U6 RNA Pol III promoter, and more preferably the human U6snRNA Pol III promoter, which has been used previously for expression of short, defined ribozyme transcripts in human cells (Bertrand, E. et al., 1997; Good, P. D. et al., 1997). The U6 Pol III promoter and its simple termination sequence (four to six uridines) were found to express siRNAs in cells. Appropriately selected interfering RNA or siRNA encoding sequences can be inserted into a transcriptional cassette, providing an optimal system for testing endogenous expression and function of the RNA molecules.

In a preferred embodiment, the mammalian cells are human cells. However, it is also understood that the invention may be carried out in other target cells, such as other types of vertebrate cells or eukaryotic cells.

In accordance with the invention, effective expression of siRNAs providing DNA methylation of sites on the promoter region of the RASSF1A gene was demonstrated in human cells.

The above results were achieved using a human U6 pol III promoter to express an appropriate 21 nucleotides siRNA in human cells. Methylation of the promoter region of the RASSF1A gene also was achieved using a 28 nucleotides siRNA expressed in mammalian cells.

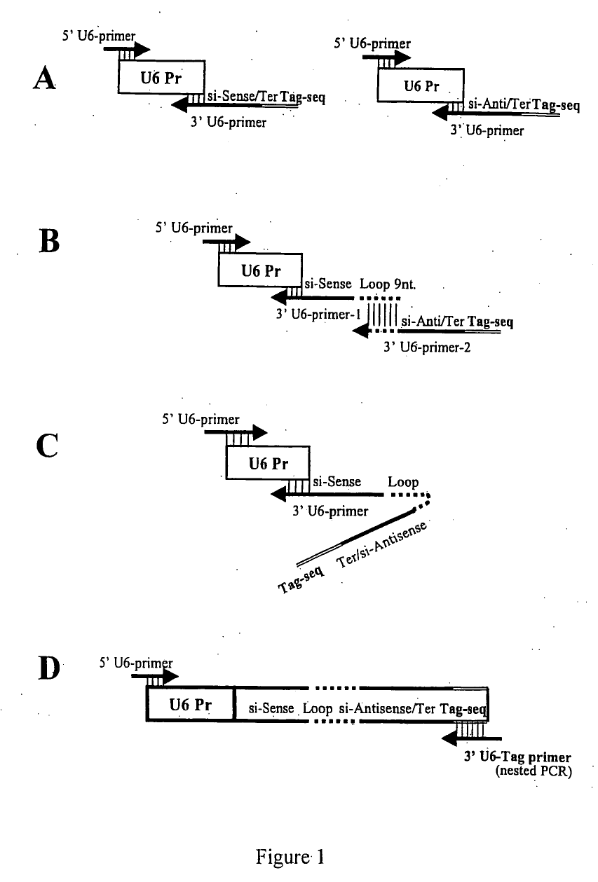

The procedure for a PCR-based approach is depicted schematically in FIG. 1 and illustrated in Example 1. In one embodiment, a universal primer that is complementary to the 5′ end of the human U6 promoter is used in a PCR reaction along with a primer(s) complementary to the 3′ end of the promoter, which primer harbors appended sequences which are complementary to the sense or antisense siRNA genes (FIG. 1A). The sense or antisense sequences are followed by a transcription terminator sequence (Ter), which is preferably a stretch of 4-6 deoxyadenosines, and more preferably a stretch of 6 deoxyadenosines, and by a short additional “stuffer-tag” sequence that may include a restriction site for possible cloning at a later stage. The resulting PCR products include the U6 promoter sequence, the siRNA sense or antisense encoding sequence, a terminator sequence, and a short tag sequence at the 3′ terminus of the product.

In another embodiment, both the sense and antisense sequences can be included in the same cassette (FIGS. 1B, 1C and 1D). In this case a nucleotide loop, preferably containing 9 nucleotides, is inserted between the sense and antisense siRNA sequences. The resulting single PCR product includes the U6 promoter, the siRNA sense and antisense encoding sequences in the form of a stem-loop, the Pol III terminator sequence, and the “stuffer” tag sequence (FIG. 1D). To construct this cassette two 3′ primers are used. The first PCR reaction employs the 5′ U6 universal (or “common”) primer and a 3′ primer complementary to a portion of the U6 promoter, followed by sequences complementary to the siRNA sense encoding sequence and the 9 nt. loop (FIG. 1B). Preferably one microliter of this first reaction is re-amplified in a second PCR reaction that employs the same 5′ U6 primer and a 3′ primer harboring sequences complementary to the 9 nt. loop appended to the antisense strand, Ter and “stuffer” tag sequence (FIG. 1B).

In another embodiment, a one step PCR reaction is conducted with a single 3′ primer that harbors the sense, loop, antisense, Ter and “stuffer’ tag sequences (FIG. 1C).

PCR conditions are relatively standard for all siRNA genes since the regions complementary to the U6 promoter do not change. For the construction of several cassettes, optimal amplification can be achieved in each case using 1 minute for each PCR step and 55° C. as annealing temperature. For direct tranfections and testing of the PCR amplified siRNA genes, the 5′ termini of the PCR primers can be phosphorylated using a DNA polynucleotide kinase and non-radioactive ATP. This modification results in enhanced efficacy of the PCR products perhaps, by stabilizing them intracellularly.

Once the PCR reaction is completed, the products can be column purified from the primers, e.g., via a gel filtration column or by excising them directly from a gel following electrophoresis. The purified products can be applied to cells following cationic liposome encapsidation and/or standard transfection procedures, such as those described below and in co-pending Application Serial No. 60/356,127, filed on Feb. 14, 2002, which is incorporated herein by reference.

Cells whose genome includes the gene containing the target sequence or sequences can be stably transfected with the expression construct(s) expressing siRNAs. Transfection can be achieved by methods known in the art and further described in the Examples below. Cells then can be selected and monitored either in mixed population or clones of transfected cells.

Various methods can be employed to determine whether methylation was successful. For example, in the mixed cell population, genomic DNA can be treated with bisulfite, which changes unmethylated, but not methylated, cytosines to thymidines. PCR primers specific for either methylated or unmethylated nucleotides can be used in PCR reactions with a methylation-specific PCR assay, such as the MSP assay described in Herman, et al. This assay is sensitive and specific for methylation of virtually any block of CpG sites in a CpG island. Restriction analysis with an enzyme that recognizes only the methylated sequence can be used to confirm the presence of methylated sites in the gene. Finally, a negative control can be introduced by analyzing DNA from cells expressing a mutated or random siRNA and determining whether methylation occurred.

In light of the preceding description, one of ordinary skill in the art can practice the invention to its fullest extent. The following examples, therefore, are merely illustrative and should not be construed to limit in any way the invention as set forth in the claims, which follow.

### EXAMPLE 1

This example demonstrates expression of short hairpin RNAs that are complementary to regions of a human tumor suppressor gene RASSF1A. The consequences of this expression were monitored by determining the patterns of DNA methylation in the promoter and part of the coding region of this gene, which is also susceptible to methylation in cancer cells. The DNA sequence of the RASSF1A gene is depicted below:

RASSF1A Promoter:
ggggctctgc gagagcgcgc ccagccccgc cttcgggccc
cacagtccct gcacccaggt ttccattgcg cggctctcct
cagctccttc ccgccgccca gtctggatcc tgggggaggc
gctgaagtcg gggcccgccc tgtggccccg cccggcccgc
gcttgct [symbol] gcccaaagcc

RASSF1A transcript:
agcgaagcac gggcccaaCC GGgccatgtc gggggagcct
gagctcattg agctgcggga gctggcaccc gctgggcgcg
ctgggaaggg ccgcacccgg ctggagcgtg ccaacgcgct
gcgcatcgcg cggggcaccg cgtgcaaccc cacacggcag
ctggtccctg gccgtggcca ccgcttccag cccgcggggc
ccgccacgca cacgtggtgc gacctctgtg gcgacttcat
ctggggcgtc gtgcgcaaag gcctgcagtg cgcgcgtgag
tagtggcccc gcgcgcctac

agc is where transcription probably starts

atg is the methionine codon

The bolded sequences were targeted by siRNAs of the invention.

Polymerase Chain Reaction

PCR reactions are performed using a plasmid containing the human U6 promoter as template to yield U6 transcription cassettes expressing siRNAs. The 5′ oligonucleotide (5′U6 universal primer) is complementary to 29 nucleotides at the 5′ end of the U6 promoter (bold italics indicate the nucleotides complementary to those on the promoter).

5′U6 Mlu primer:
5′AATCGA ACGCGT [symbol] 3′
Mlu I U6

This U6 common 5′ primer, used for all PCR steps, binds to the 5′ end of the U6 promoter and includes an Mlu I restriction site for cloning purposes. The 3′ oligonucleotides, which contain either the sense, antisense, or both siRNA-coding sequences (siDNAs), are depicted in FIG. 1 and described herein. The last 20 nucleotides at the 3′ end of all 3′ PCR primers are complementary to the last 20 nucleotides of the U6 promoter which is: 5′GTGGAAAGG ACGAAACACCG3′. All PCR reactions were carried out as follows: 1 min. at 94° C., 1 min. at 55° C. and 1 min. at 72° C. for 30 cycles. The PCR products can be directly transfected into cells (e.g., with prior cloning into an expression vector), in which event the PCR primers can be kinased with non-radioactive ATP prior to amplification and purified on Quiagen columns prior to using them in the PCR reactions. The PCR products also can be purified on Quiagen columns.

The 3′ primers used to make siRNA expression cassettes are depicted below:

Primers used to make PCR products encoding siRNA's complementary to the promoter region of the RASSF1A gene:

```
3′PR 1
5′CTACACAAA GGCGGGCCCCGACTTCAGCG C
          loop si-sense +1
GGTGTTTCGTCCTTTCCACAA 3′
                  U6
3′PR 2
5′AACTC GAATTC AAAAAA GCGCTGAAGTCGGGGCCCGCC
                  EcoRI Ter. si-antisense
CTACACAAA 3′
    Loop
```

Primers used to make PCR products encoding siRNA's complementary to the transcribed region of the RASSF1A gene:

```
′TR 1
5′CTACACAAA CGACATGGCCCGGTTGGGCC C
        loop si-sense +1
GGTGTTTCGTCCTTTCCACAA 3′
                  U6
3′TR 2
5′AACTC GAATTC AAAAAA GGGCCCAACCGGGCCATGTCG
                    EcoRI Ter. si-antisense
CTACACAAA 3′
    Loop
```

### EXAMPLE 2

HeLa cells, which include in their genome the RASSF1A gene, were stably transfected with the siRNA expression constructs produced by the method shown above. The final siRNAs-containing PCR products were digested with MluI and EcoRI and cloned in the same sites of the pcDNA3.1 vector (Invitrogen) for expression in the mammalian cells. Digestion of pcDNA3.1 with MluI and EcoRI allows the replacement of the CMV promoter with the U6 siRNA cassettes. The Neomycin gene is the marker gene for selection in mammalian cells. Cells were selected for G418 resistance. Cells were monitored either in mixed population or clones of transfected cells.

Stable cell lines expressing all different siRNAs and 8 individual single clones for each of the siRNA expressing cells have thus far been obtained.

Examples 3-6 illustrate methods used to determine whether methylation was successful.

### EXAMPLE 3

In the mixed cell population, genomic DNA was isolated and treated with bisulfite, which changes unmethylated cytosines to thymidines. Methylated cytosines remain as cytosine. Thus, if the siRNAs direct methylation of the targeted sequences of the RASSF1A shown in Example 1, these DNAs will not be modified by bisulfite in the methylated region.

### EXAMPLE 4

PCR primers specific for either methylated or unmethylated nucleotides were used in PCR reactions in accordance with the Methylation-specific PCR assay (MSP assay) described in Herman et al. Results showed that the siRNA that targets the promoter region and the siRNA that targets the RASSF1A transcript, were directing methylation of the RASSF1A gene. The MSP assay is sensitive and specific for methylation of virtually any block of CpG sites in a CpG island. The assay uses primers designed to distinguish methylated from unmethylated DNA in bisulfite-modified DNA, taking advantage of the sequence differences resulting from bisulfite modification. Unmodified DNA or DNA incompletely reacted with bisulfite can also be distinguished, since marked sequence differences exist between these DNAs.

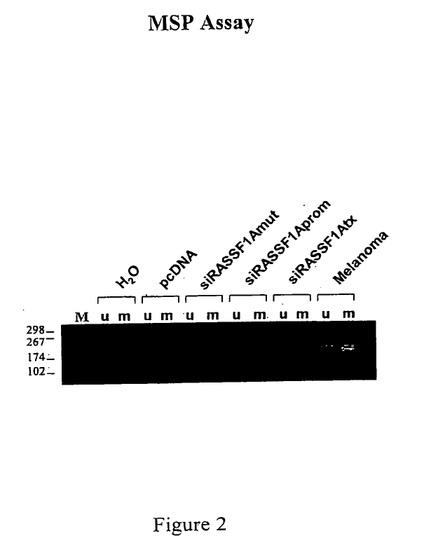

FIG. 2 shows results of the MSP analysis of the RASSF1A promoter in siRNA transfected cells. In the figure, H₂O represents a water control used in the PCR reactions. The following additional abbreviations were also used:

pcDNA: Cells transfected only with the vector (no siRNA)

siRASSF1Amut: Cells transfected with the mutant siRNA vector

siRASSF1Aprom: Cells transfected with the siRNA vector directed against the RASSF1A promoter sequences

siRASSF1Atx: Cells transfected with the siRNA vector directed against the RASSF1A transcript

Melanoma: a control for RASSF1A methylation. This is DNA from a melanoma tumor, which is methylated in the RASSF1A promoter.

M, size markers

m, MSP done with primers specific for a methylated RASSF1A promoter

u, MSP done with primers specific for an unmethylated RASSF1A promoter

The following primers were used in the MSP reaction: methylated DNA-specific primers, M210 (5′ GGGTTTTGCGAGAGCGCG 3′) and M211 (5′GCTAACAAACGCGAACCG 3′) or unmethylated DNA-specific primers UM240 (5′ GGGGTTTTGTGAGAGTGTGTTTAG 3′) and UM241 (5′ TAAACACTAACAAACACAAACCAAAC 3′) (Liu, L. et al., 2002).

### EXAMPLE 5

Restriction analyses with an enzyme that recognizes only the methylated sequence (BstU1), also confirmed the presence of methylated sites in the RASSF1A gene.

### EXAMPLE 6

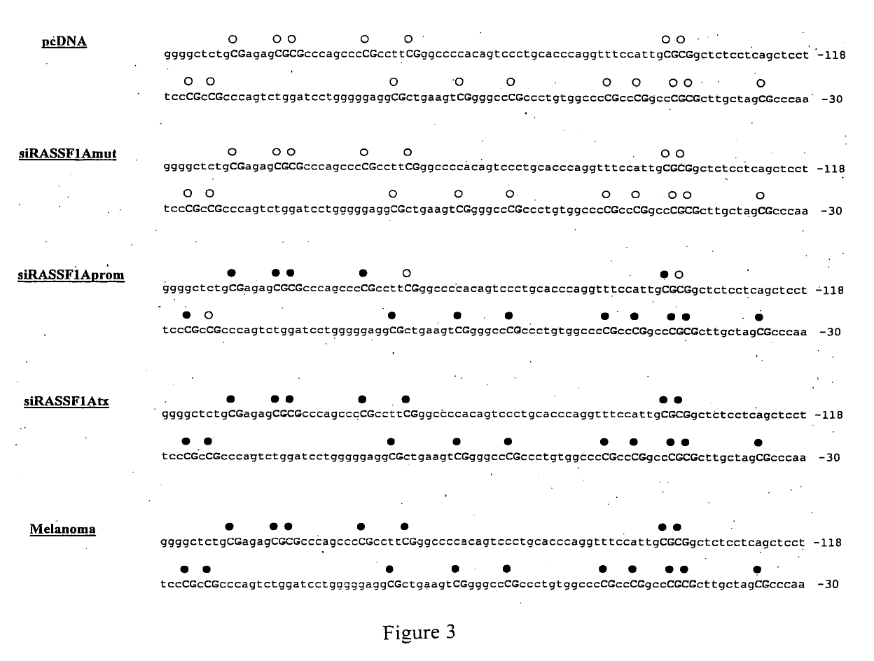

Specific deoxynucleotide primed sequencing revealed that 14 out of 17 potential methylation sites analyzed in the RASSF1A gene were methylated in cell populations expressing the siRNA directed against the RASSF1A promoter, and 17 out of 17 sites were methylated in cells expressing the siRNA directed against a CpG island in the RASSF1A transcript. Results are shown in FIG. 3. The level of methylation in the promoter region was higher in some of the single clones analyzed. Specific integration sites of siRNAs in the cellular genome (by using the appropriate delivering vector) could be used to achieve complete promoter methylation.

Sequence data were obtained by sequencing of the PCR products obtained from the MSP reactions of Example 4 (FIG. 2). In FIG. 3, sample designation is the same as in FIG. 2. FIG. 3 shows the RASSF1A promoter sequence relative to the ATG translation start site (i.e. −30 indicates 30 nucleotides upstream). Open circles represent unmethylated cytosines at CG sequences. Closed circles indicate methylated cytosines at CG sequences.

### EXAMPLE 7

As a negative control, DNA was extracted from cells expressing a mutated siRNA, was analyzed, and showed no effects on the methylation of the RASSF1A gene. In this analysis, PCR products were produced as described in Example 1, but using the 3′ primers shown below. For the mutant there were two transversions (CCGG to GGCC) and one transition (C to T) to make sure it would be inactive.

Mutant primers against transcribed region:

```
3′MT 1
                            (c) (cc gg)
5′CTACACAAA CGATATGGCGGCCTTGGGCC C
          loop si-sense +1
GGTGTTTCGTCCTTTCCACAA 3′
                  U6
3′MT 2
5′AACTC GAATTC AAAAAA GGGCCCAAGGCCGCCATATCG
                  EcoRI Ter. si-antisense
CTACACAAA 3′
    Loop
```

### EXAMPLE 8

RASSF1A intracellular expression in stable clones and cell populations is reduced when the cells are transfected with shRNAs directed against promoter sequences.

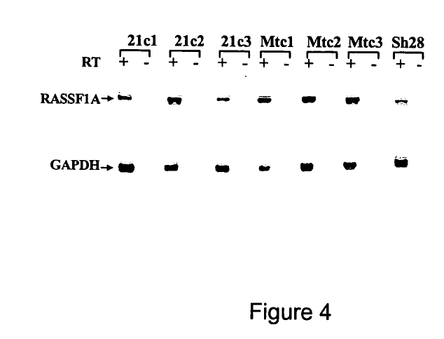

FIG. 4 shows the reduction of RASSF1A RNA transcripts detected by reverse transcriptase PCR (RT-PCR) reactions. Hela cells were transfected with shRNAs directed against promoter sequences of RASSF1A. Cells were collected after 48-56 hr. and the RNA was extracted using RNA STAT60 as suggested by the manufacturer. Quantitative PCR reactions were performed by preparing 100 μl PCR mixes containing standard PCR buffer, dNTPs, 1 μl of each RNA sample, and two 3′ primers specific to either the RASSF1A transcript or to the GAPDH cellular gene. GAPDH is used as an internal control to verify the integrity and amount of RNA analyzed in each reaction. After the samples were heated at 80° C. for 1 minute and slow cooled to room temperature, they were thoroughly mixed and divided into two 50 μl aliquots. 1-2 units of reverse transcriptase were added to half of the reactions while the other half were used as controls to exclude DNA contaminations. All samples were placed at 37° C. for 5 minute to complete the extension reactions. Following the extensions (and cDNA synthesis) the samples were thoroughly mixed and divided once again into two 25 μl aliquots. The specific 5′ primers for the RASSFLA or the GAPDH were added to the 25 μl aliquots and the PCR reactions were completed as for the methylation-specific PCR assay.

As shown in FIG. 4, representative clonal cell lines from cells transfected with the 21 nucleotides shRNAs directed against the RASSF1A promoter (21c1, 21c2, 21c3), and the Hela cell population transfected with a 28 nucleotides shRNA (sh28) were analyzed for decreased RNA expression. Clonal cell lines tranfected with the shRNA mutant (Mtc1, Mtc2, Mtc3) were also analyzed as controls. After normalization with the GAPDH internal control, a clear and specific RASSF1A RNA down-regulation can be detected in two of the three clones expressing shRNA directed against promoter sequences, but in none of the mutant shRNA clones used as controls. The -RT controls showed no DNA contamination.

These results indicate that specific shRNA methylation of the RASSF1A promoter results in down-regulation of the intracellular RASSF1A transcripts.

### EXAMPLE 9

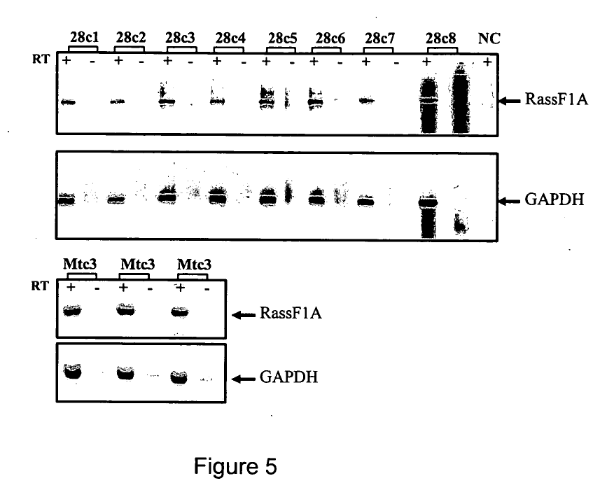

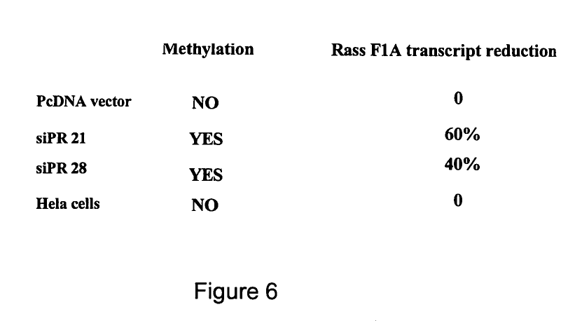

Several clonal Hela cell lines transfected with 28 nucleotides shRNAs directed against the promoter sequences were analyzed by Reverse Transcriptase dependent PCRs as described in Example 8. The results shown in FIG. 5 show decrease expression of RASSFLA transcripts in many of the clones analyzed. Similar results were obtained by expressing the shRNAs from lentiviral vector backbones (not shown), which may be the method of choice (but not the only method) for long-term expression of shRNAs and gene silencing. The results obtained with the clonal cell lines transfected with the various shRNAs are summarized in FIG. 6.

The above demonstrates the invention's utility for, among other things, designing and using siRNAs to direct DNA methylation in either a promoter region or certain coding region of a gene. Directing promoter methylation of a gene by targeting siRNAs against CpG islands of RNA transcripts should be a potent inhibitor of intracellular gene expression.

While the invention has been disclosed by reference to the details of preferred embodiments of the invention, it is to be understood that the disclosure is intended in an illustrative rather than a limiting sense, as it is contemplated that modifications will readily occur to those skilled in the art, within the spirit of the invention and the scope of the appended claims.

### References

1. Hammond, S. M., Bernstein, E., Beach, D. & Hannon, G. J. An RNA-directed nuclease mediates post-transcriptional gene silencing in Drosophila cells. Nature 404, 293-296 (2000).

2. Fire, A. RNA-triggered gene silencing. Trends Genet 15, 358-363 (1999).

3. Sharp, P. A. RNA interference—2001. Genes Dev 15, 485-490 (2001).

4. Elbashir, S. M. et al. Duplexes of 21-nucleotide RNAs mediate RNA interference in cultured mammalian cells. Nature 411, 494-498 (2001a).

5. Caplen, N. J., Parrish, S., Imani, F., Fire, A. & Morgan, R. A. Specific inhibition of gene expression by small double-stranded RNAs in invertebrate and vertebrate systems. Proc Natl Acad Sci USA 98, 9742-9747 (2001).

6. Bertrand, E. et al. The expression cassette determines the functional activity of ribozymes in mammalian cells by controlling their intracellular localization. Rna 3, 75-88 (1997).

7. Good, P. D. et al. Expression of small, therapeutic RNAs in human cell nuclei. Gene Ther 4, 45-54 (1997).

8. Scherr, M. & Rossi, J. J. Rapid determination and quantitation of the accessibility to native RNAs by antisense oligodeoxynucleotides in murine cell extracts. Nucleic Acids Res 26, 5079-5085 (1998).

9. Scherr, M. et al. Detection of Antisense and Ribozyme Accessible Sites on Native mRNAs: Application to NCOA3 mRNA. Mol Ther 4, 454-460 (2001).

10. Lee, N. S. et al. Functional colocalization of ribozymes and target mRNAs in Drosophila oocytes. Faseb J 15, 2390-2400 (2001).

11. Bernstein, E., Caudy, A. A., Hammond, S. M. & Hannon, G. J. Role for a bidentate ribonuclease in the initiation step of RNA interference. Nature 409, 363-366 (2001).

12. Clemens, J. C. et al. Use of double-stranded RNA interference in Drosophila cell lines to dissect signal transduction pathways. Proc Natl Acad Sci USA 97, 6499-6503 (2000).

13. Lipardi, C., Wei, Q. & Paterson, B. M. RNAi as Random Degradative PCR. siRNA Primers Convert mRNA into dsRNAs that Are Degraded to Generate New siRNAs. Cell 107, 297-307 (2001).

14. Sijen, T. et al. On the Role of RNA Amplification in dsRNA-Triggered Gene Silencing. Cell 107, 465-476 (2001).

15. Elbashir, S. M., Lendeckel, W. & Tuschl, T. RNA interference is mediated by 21- and 22-nucleotide RNAs. Genes Dev 15, 188-200 (2001b).

16. Ui-Tei, K., Zenno, S., Miyata, Y. & Saigo, K. Sensitive assay of RNA interference in Drosophila and Chinese hamster cultured cells using firefly luciferase gene as target. FEBS Lett 479, 79-82 (2000).

17. Wianny, F. & Zernicka-Goetz, M. Specific interference with gene function by double-stranded RNA in early mouse development. Nat Cell Biol 2, 70-75 (2000).

18. Kennerdell, J. R. & Carthew, R. W. Use of dsRNA-mediated genetic interference to demonstrate that frizzled and frizzled 2 act in the wingless pathway. Cell 95, 1017-1026 (1998).

19. Fire, A. et al. Potent and specific genetic interference by double-stranded RNA in Caenorhabditis elegans. Nature 391, 806-811 (1998).

20. Oelgeschlager, M., Larrain, J., Geissert, D. & De Robertis, E. M. The evolutionarily conserved BMP-binding protein Twisted gastrulation promotes BMP signalling. Nature 405, 757-763 (2000).

21. Svoboda, P., Stein, P., Hayashi, H. & Schultz, R. M. Selective reduction of dormant maternal mRNAs in mouse oocytes by RNA interference. Development 127, 4147-4156 (2000).

22. Dalmay, T., Horsefield, R., Braunstein, T. H. & Baulcombe, D. C. SDE3 encodes an RNA helicase required for post-transcriptional gene silencing in Arabidopsis. Embo J 20, 2069-2078 (2001).

23. Cogoni, C. & Macino, G. Posttranscriptional gene silencing in Neurospora by a RecQ DNA helicase. Science 286, 2342-2344 (1999).

24. Nykanen, A., Haley, B. & Zamore, P. D. ATP requirements and small interfering RNA structure in the RNA interference pathway. Cell 107, 309-321 (2001).

25. Smardon, A. et al. EGO-1 is related to RNA-directed RNA polymerase and functions in germ-line development and RNA interference in C. elegans. Curr Biol 10, 169-178 (2000).

26. Lee, N. S., Bertrand, E. & Rossi, J. mRNA localization signals can enhance the intracellular effectiveness of hammerhead ribozymes. Rna 5, 1200-1209 (1999).

27. Hamilton, A., et al., Two classes of short interfering RNA in RNA silencing. EMBO Journal 21, 4671-4679 (2002).

28. Lee, N. S., et al. Expression of small interfering RNAs targeted against HIV-1 rev transcripts in human cells. Nat Biotechnol. 20(5), 500-505 (2002).

29. Miyagishi, M. and K. Taira. U6 promoter driven siRNAs with four uridine 3′ overhangs efficiently suppress targeted gene expression in mammalian cells. Nat Biotechnol. 20(5), 497-500 (2002).

30. Paul, C. P., et al. Effective expression of small interfering RNA in human cells. Nat Biotechnol. 20(5), 505-508 (2002).

31. Brummelkamp, T. R., et al. A system for stable expression of short interfering RNAs in mammalian cells. Science 296(5567), 550-553 (2002).

32. Ketting, R. F., et al. Dicer functions of RNA interference and in synthesis of small RNA involved in developmental timing in C. elegans. Genes Dev. 15(20), 2654-9 (2001).

33. Yu, J. Y., et al. RNA interference by expression of short-interfering RNAs and hairpin RNAs in mammalian cells. PNAS USA 99(9), 6047-52 (2002).

34. Holen, T., et al. Positional effects of short interfering RNAs targeting the human coagulation trigger Tissue Factor. Nucleic Acids Res. 30(8), 1757-66 (2002).

35. Herman, J. G., et al. Methylation-specific PCR: A novel PCR assay for methylation status of CpG islands. PNAS USA 93, 9821-9826 (1996).

36. Liu, L., et al. Frequent hypermethylation of the RASSF1A gene in prostate cancer, Oncogene 21, 6835-6840 (2002).

What is claimed is:

1. A method for methylating a gene of interest in a mammalian cell comprising:
exposing said cell to an siRNA molecule which is specific for a target sequence in said gene of interest, wherein said siRNA directs methylation of said gene of interest.

2. The method of claim 1, wherein said target sequence is located in a promoter region of said gene of interest.

3. The method of claim 1, wherein said target sequence is located in a coding region of said gene of interest.

4. The method of claim 1, wherein said siRNA directs methylation of a promoter region of said gene of interest.

5. The method of claim 3, wherein said target sequence comprises a CpG island.

6. The method of claim 1, wherein said siRNA contains about 19-28 base pairs.

7. The method of claim 6, wherein said siRNA contains about 21 base pairs.

8. The method of claim 1, wherein said mammalian cell is a human cell.

9. The method of claim 1, wherein said gene of interest is an infectious agent gene.

10. The method of claim 8, wherein said infectious agent is viral.

11. The method of claim 1, wherein said cell is exposed to said siRNA by introducing into said cell DNA sequences encoding a sense strand and a antisense strand of said siRNA, wherein said siRNA is expressed in the cell.

12. The method of claim 11, wherein said introducing is accomplished using at least one vector.

13. The method of claim 12, wherein said vector is a plasmid vector.

14. The method of claim 12, wherein said vector is a viral vector.

15. The method of claim 14, wherein said viral vector is a retroviral vector, a lentiviral vector, or an adenoviral vector.

16. The method of claim 12, wherein said vector is an adeno-associated vector.

17. The method of claim 11, wherein said introducing takes place in vivo.

18. The method of claim 11, wherein said introducing takes place in vitro.

19. The method of claim 11, wherein said introducing is achieved via transformation, transduction, transfection, or infection.

20. The method of claim 11, wherein said introducing is achieved via a liposome.

21. The method of claim 11, wherein said DNA sequences are generated by PCR.

22. The method of claim 1, wherein said gene is a RASSF1 gene.

23. The method of claim 12, wherein said DNA sequences are in the same vector.

24. The method of claim 12, wherein said DNA sequences are in separate vectors.

25. The method of claim 1, wherein said method causes inactivation of said gene of interest.

26. The method of claim 1, wherein said method causes activation of said gene of interest.
